(19) United States
(12) Patent Application Publication
(10) Pub. No.: US 2020/0247065 A1
(43) Pub. Date: Aug. 6, 2020

(54) SEALING BAR AND METHOD FOR CONNECTING SURGICAL GRADE MATERIALS

(71) Applicant: Exact Medical Manufacturing Inc., Lancaster, NY (US)

(72) Inventors: David Power, Elma, NY (US); Ryan Power, Lancaster, NY (US)

(21) Appl. No.: 16/266,247

(22) Filed: Feb. 4, 2019

(51) Int. Cl. B29C 65/20; A61B 46/00; B29C 65/78; B29C 65/00; A41D 13/12

(52) U.S. Cl. CPC B29C 65/20; A61B 46/40; B29C 65/7802; B29L 2031/48; B29C 66/729; A41D 13/1236; B29C 66/43

(57) ABSTRACT

A sealing bar for creating an Association for the Advancement of Medical Instrumentation (AAMI) rated seal between a first layer of AAMI rated material and a second layer of AAMI rated material, comprising a first component including a first width and a first height, a second component connected to the first component, the second component including a second width, a second height, and an engaging surface, wherein the engaging surface is operatively arranged to engage the first and second layers of AAMI rated materials and form the seal therebetween.

### FIELD

The present disclosure relates to the field of medical materials, and more particularly, to surgical gowns and drapes, and even more particularly, to a method of forming a seal between two pieces of surgical materials that provides the same level of protection as that of the surrounding material.

### BACKGROUND

Poly spun bond materials are known. Because the cost of manufacture is relatively low, such poly spun bond materials have a wide variety of possible applications. Poly spun bond materials are often used in surgical drapes and garments. However, materials that are used for surgical drapes and garments are governed by a set of standards and test methods that all manufacturers must meet. These standards are established by the Association for the Advancement of Medical Instrumentation (AAMI). Specifically, American National Standards Institute (ANSI)/AAMI PB70:2012 is the standard that establishes a classification system (Levels 1-4) for protective apparel used in health care facilities, including surgical gowns, based on liquid barrier performance using standardized test methods. Of Levels 1-4, Level 1 is the least protective and Level 4 is the most protective. However, connecting two pieces of AAMI Level 4 rated materials by creating a seam or seal between the two pieces may result in the loss of the AAMI Level 4 rating. This may be due to the degradation of the AAMI Level 4 rated materials during the creation of the seam or seal (e.g., from heat or pressure).

Thus, there is also a long felt need for process for connecting two pieces of AAMI Level 4 rated materials with a seal that maintains the AAMI Level 4 rating. There is also a long felt need for a sealing bar that can be used during the process that creates an AAMI Level 4 rated seal between the two pieces of AAMI level 4 materials. There is also a long felt need for a material assembly (e.g., surgical gown or drape) having two pieces of material connected together by a seal, which maintains an overall AAMI Level 4 rating.

### SUMMARY

According to aspects illustrated herein, there is provided a sealing bar for creating an Association for the Advancement of Medical Instrumentation (AAMI) rated seal between a first layer of AAMI rated material and a second layer of AAMI rated material, comprising a first component including a first width and a first height, a second component connected to the first component, the second component including a second width, a second height, and an engaging surface, wherein the engaging surface is operatively arranged to engage the first and second layers of AAMI rated materials and form the seal therebetween.

According to aspects illustrated herein, there is provided a method of connecting a first layer of Association for the Advancement of Medical Instrumentation (AAMI) rated material and a second layer of AAMI rated material, the method comprising arranging the first layer of AAMI rated material on a first platen, the first layer of AAMI rated material including a first surface arranged facing toward the first platen and a second surface arranged facing away from the first platen, arranging the second layer of AAMI rated material on the first layer of AAMI rated material, the second layer of AAMI rated material including a third surface arranged facing toward the second surface and a fourth surface facing away from the second surface, engaging a sealing bar with the first and second layers of AAMI rated material, the sealing bar comprising an engaging surface arranged to contact the fourth surface, applying a preset amount of pressure to the sealing bar, heating the sealing bar to a predetermined temperature, and forming an AAMI rated seal between the first layer of AAMI rated material and the second layer of AAMI rated material.

According to aspects illustrated herein, there is provided an Association for the Advancement of Medical Instrumentation (AAMI) rated surgical gown or drape comprising at least a first layer of AAMI rated material and a second layer of AAMI rated material, the AAMI rated surgical gown or drape formed using the steps of arranging the first layer of AAMI rated material on a first platen, the first layer of AAMI rated material including a first surface arranged facing toward the first platen and a second surface arranged facing away from the first platen, arranging the second layer of AAMI rated material on the first layer of AAMI rated material, the second layer of AAMI rated material including a third surface arranged facing toward the second surface and a fourth surface facing away from the second surface, engaging a sealing bar with the first and second layers of AAMI rated material, the sealing bar comprising an engaging surface arranged to contact the fourth surface, applying a preset amount of pressure to the sealing bar, heating the sealing bar to a predetermined temperature, and forming an AAMI rated seal between the first layer of AAMI rated material and the second layer of AAMI rated material.

The present disclosure comprises a heat-sealed biological barrier material capable of resisting penetration by viral vectors, and an apparatus and method for generating a heat-sealed seam or coupling between at least two pieces of barrier material. The present disclosure may comprise 1) a die; 2) a process by which the sealing of fabrics occurs using said die; and 3) a product by process (sealed fabrics).

The die comprises a configuration, dimension, and composition to allow for the creation of a non-tearing coupling between fabrics or materials that is at least as repellant to biological penetration as the surrounding material. The die is configured to have a T-shape cross-section and of a composition suitable for controllable, uniform heat conduction and delivery at the contact point, wherein the seal is generated. The dimension of the contact point is further tailored to allow for a seal of sufficient width to prevent tearing and maintain the protective integrity of the surrounding fabrics.

The process utilizes the specially designed, heated sealing die operatively arranged within a sealing press to generate a biologically impermeable seam that is at least as repellant to biological penetration as the surrounding material. The process of heat sealing the biologically impenetrable fabrics begins when at least two pieces of the fabric are aligned and placed on top of each other, wherein the inward-facing surfaces are exposed and outward-facing surfaces are arranged transversely such that they are facing and each other. The aligned fabrics are then placed in the heating press wherein the specially-designed die is lowered onto the fabrics and pressure and heat are applied. The cross-section of the point of contact is of a width sufficient to provide a secure seal, in the form of a seam or coupling while maintaining the permeability rating of the constituent fabric. The coupled fabrics may then be opened or inverted to expose the final outward-facing surfaces coupled by the outwardly exposed, biologically impenetrable coupling or seal.

The product resulting from the process exhibits uniform impenetrability at all points, including at the seal. This particular seal and surrounding material are intended to be impenetrable to viral vectors, proteins, and blood and thus have a “Level 4” rating by the AAMI.

These and other objects, features, and advantages of the present disclosure will become readily apparent upon a review of the following detailed description of the disclosure, in view of the drawings and appended claims.

### BRIEF DESCRIPTION OF THE DRAWINGS

Various embodiments are disclosed, by way of example only, with reference to the accompanying schematic drawings in which corresponding reference symbols indicate corresponding parts, in which:

FIG. 1A is a front elevational view of a press assembly;

FIG. 1B is a side elevational view of the press assembly shown in FIG. 1A;

FIG. 1C is a top elevational view of the press assembly shown in FIG. 1A;

FIG. 2 is a cross-sectional view of a sealing bar taken generally along line 2-2 in FIG. 1A;

FIG. 3A is a perspective view of a material assembly;

FIG. 3B is a perspective view of a material assembly;

FIG. 3C is a perspective view of a material assembly; and,

FIG. 3D is a perspective view of a material assembly.

### DETAILED DESCRIPTION

At the outset, it should be appreciated that like drawing numbers on different drawing views identify identical, or functionally similar, structural elements. It is to be understood that the claims are not limited to the disclosed aspects.

Furthermore, it is understood that this disclosure is not limited to the particular methodology, materials and modifications described and as such may, of course, vary. It is also understood that the terminology used herein is for the purpose of describing particular aspects only, and is not intended to limit the scope of the claims.

Unless defined otherwise, all technical and scientific terms used herein have the same meaning as commonly understood to one of ordinary skill in the art to which this disclosure pertains. It should be understood that any methods, devices or materials similar or equivalent to those described herein can be used in the practice or testing of the example embodiments. The assembly of the present disclosure could be driven by hydraulics, electronics, pneumatics, and/or springs.

It should be appreciated that the term “substantially” is synonymous with terms such as “nearly,” “very nearly,” “about,” “approximately,” “around,” “bordering on,” “close to,” “essentially,” “in the neighborhood of,” “in the vicinity of,” etc., and such terms may be used interchangeably as appearing in the specification and claims. It should be appreciated that the term “proximate” is synonymous with terms such as “nearby,” “close,” “adjacent,” “neighboring,” “immediate,” “adjoining,” etc., and such terms may be used interchangeably as appearing in the specification and claims. The term “approximately” is intended to mean values within ten percent of the specified value.

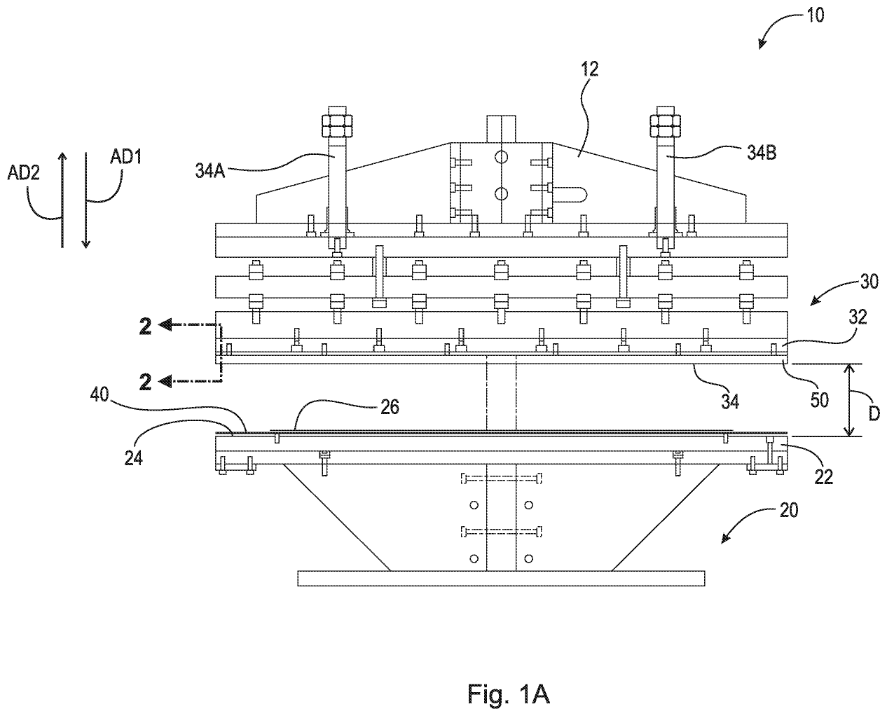

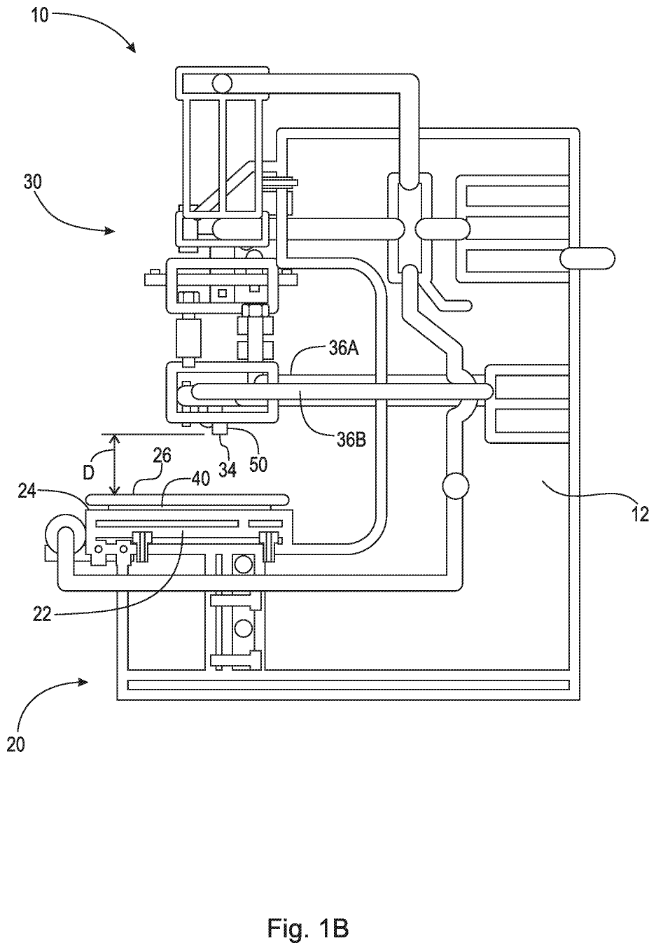

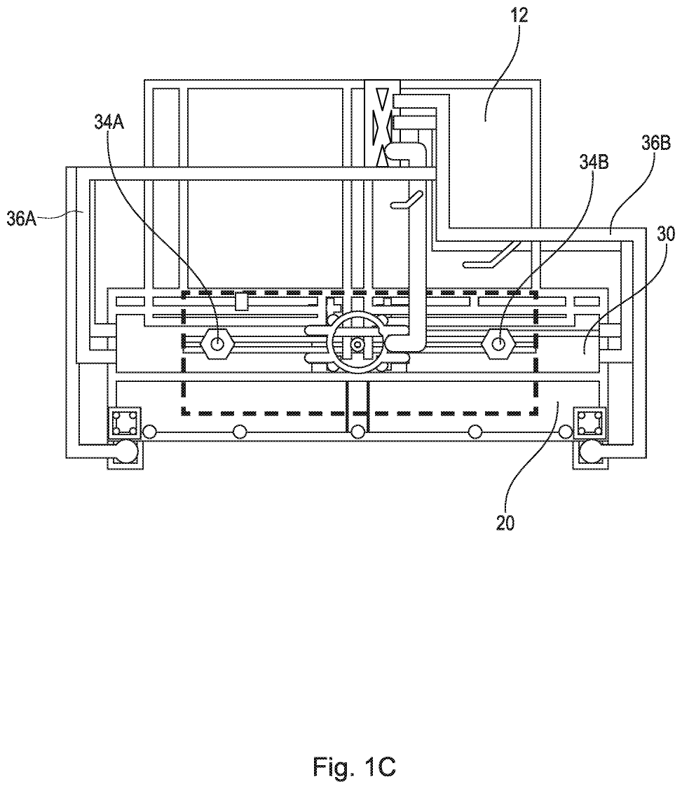

Referring now to the figures, FIG. 1A is a front elevational view of press assembly 10. FIG. 1B is a side elevational view of press assembly 10. FIG. 1C is a top elevational view of press assembly 10. Press assembly 10 generally comprises frame 12, base 20, ram 30, and sealing bar 50. The following description should be read in view of FIGS. 1A-C.

Base 20 is connected to frame 12 and comprises platen 22. Base 20 is generally arranged to be the work surface on which material assembly 40 is placed. Specifically, product assembly 40 is placed onto platen 22 such that ram 30 can engage it. In some embodiments, base 20 is fixedly secured to frame 12. In some embodiments, platen 22 comprises a planar surface. Base 20 may further comprise layer 24 connected to the top surface of platen 22. In some embodiments, layer 24 comprises TEFLON® polytetrafluoroethylene (PTFE) or another fluoropolymer of tetrafluoroethylene. In such embodiments, layer 24 may be implemented as a “non-stick” coating for platen 22. In some embodiments, layer 24 comprises 50 durometer silicon (e.g., having a thickness of 3/32 inches). In such embodiments, layer 24 may be implemented as a cushion on which material assembly 40 is arranged. Material assembly 40 is then placed on layer 24 to be assembled. An additional layer 26 may be arranged on top of material assembly 40 prior to engagement by ram 30. In some embodiments, layer 26 comprises TEFLON® PTFE or another fluoropolymer of tetrafluoroethylene and is removably arranged on material assembly 40. In some embodiments, layer 26 comprises four layers of 0.003 inch impregnated TEFLON® PTFE.

Ram 30 is connected to frame 12 and comprises platen 32, columns 34A and 34B, and sealing bar 50. In some embodiments, ram 30 is slidably connected to frame 12 and, when in a first position, is arranged such that sealing bar 50 is separated vertically from platen 22 by distance D. Ram 30 is slidable with respect to frame 12 via columns 34A and 34B. In some embodiments, columns 34A and 34B are fixedly secured to frame 12 and ram 30 slides along columns 34A and 34B. In some embodiments, columns 34A and 34B are fixedly secured to ram 30 and columns 34A and 34B and ram 30 slide with respect to frame 12 (i.e., columns 34A and 34B are slidably connected to frame 12). In some embodiments, platen 32 comprises a planar surface. Ram 30 may further comprise layer 34 connected to the bottom surface of platen 32. In some embodiments, layer 34 comprises TEFLON® polytetrafluoroethylene (PTFE) or another fluoropolymer of tetrafluoroethylene. Layer 34 may be implemented as a “non-stick” coating for platen 32. In some embodiments, layer 34 is connected to the bottom surface of sealing bar 50.

To operate press assembly 10, ram 30 is displaced in axial direction AD1 toward base 20 until sealing bar 50 engages material assembly 40. Pressure and heat are applied to sealing bar 50 until a seal is formed in material assembly 40, as will be discussed in greater detail below.

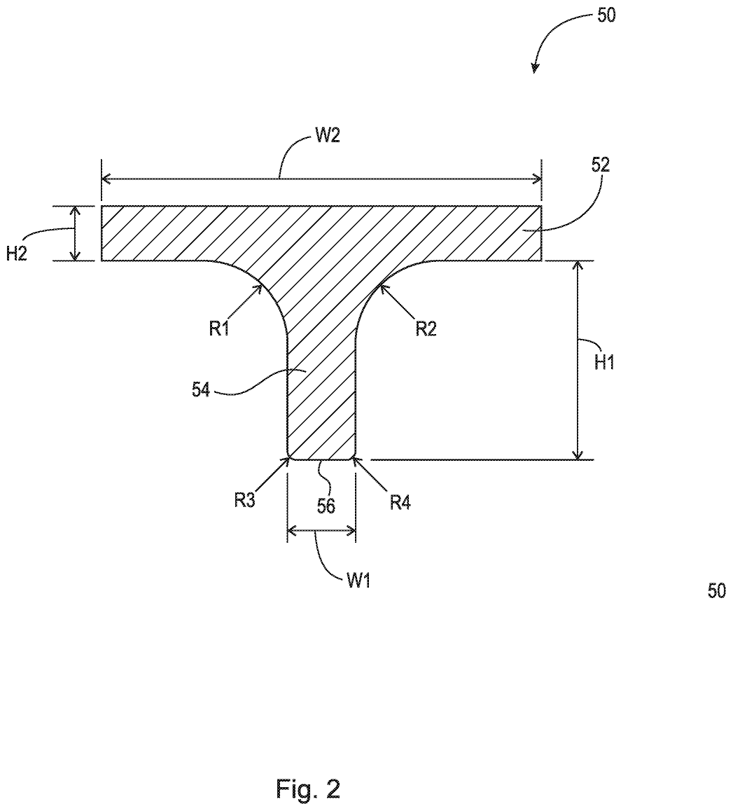

FIG. 2 is a cross-sectional view of sealing bar 50 taken generally along line 2-2 in FIG. 1A. Sealing bar 50 comprises component 52 and component 54. Component 52 is generally rectangular and includes width W2 and height 112. In some embodiments, width W2 is approximately 2.000 inches. In some embodiments, height 112 is approximately 0.250 inches.

Component 54 is generally rectangular and includes width W1 and height H1. In some embodiments, width W1 is approximately 0.250 inches. In some embodiments, height H1 is approximately 0.750 inches. Component 54 is connected to component 52. In some embodiments, component 54 is arranged perpendicular to component 52. Component 54 comprises surface 56, which is arranged to engage material assembly 40.

Sealing bar 50 further includes, where component 54 is connected to component 52, radius R1 and radius R2. In some embodiments, radius R1 is approximately 0.375 inches. In some embodiments, R2 is equal to R1. In some embodiments, R2 is greater than R1. In some embodiments, R2 is less than that R1.

Sealing bar 50 further includes, about surface 56, radius R3 and radius R4. In some embodiments, radius R3 is approximately 0.030 inches. In some embodiments, R4 is equal to R3. In some embodiments, R4 is greater than R3. In some embodiments, R4 is less than that R3.

Sealing bar 50 is specifically designed with radii R3 and R4 such that when surface 56 engages material assembly 40, an AAMI level 4 rated seal is formed. As previously discussed, sealing bar 50 is connected to platen 32 of sealing bar 50. Ram 30 is displaced in axial direction AD1 toward base 20 until sealing bar 50 engages material assembly 40. Once surface 56 is engaged with material assembly 40, a predetermined pressure is applied to sealing bar 50 and in turn material assembly 40. In some embodiments, 30 pounds per square inch of pressure is applied to sealing bar 50. Additionally, heat is applied to sealing bar 50 until surface 56 reaches a predetermined temperature or temperature within a temperature range. In some embodiments, sealing bar 50 is heated until surface 56 and material assembly 40 reaches a temperature in the range of 335 degrees Fahrenheit to 445 degrees Fahrenheit. The pressure and heat are applied to sealing bar 50, and thus material assembly 40, for a preset period of time. In some embodiments, the pressure and heat are applied to sealing bar 50, and thus material assembly 40, for 6.5 seconds. Once adequate heat and pressure are applied to sealing bar 50, a seal is formed in material assembly 40.

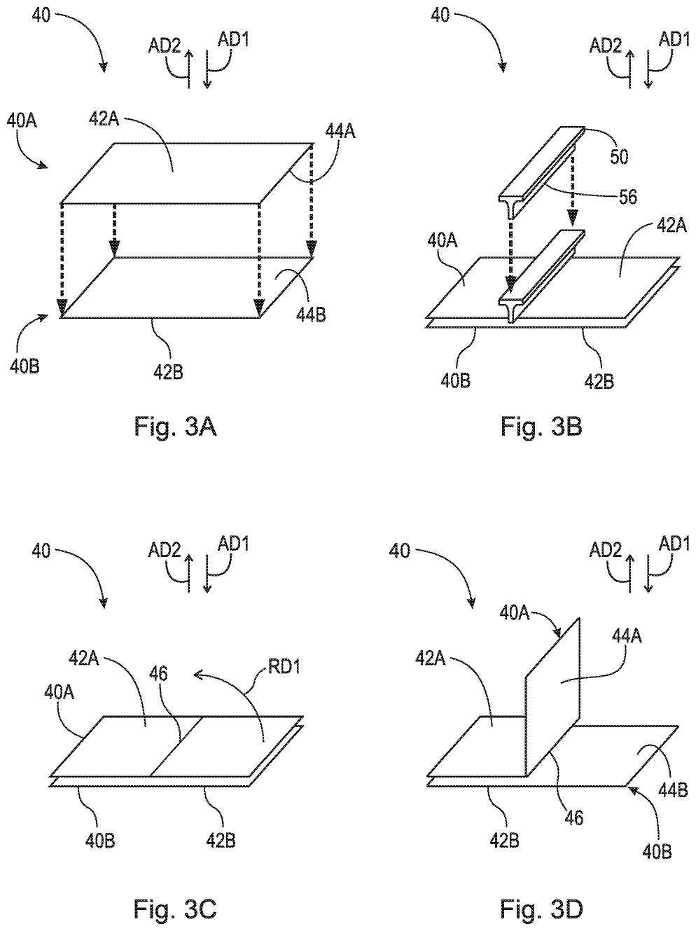

FIG. 3A is a perspective view of material assembly 40. As shown, material assembly 40 comprises layer 40A and layer 40B. Layer 40A comprises surface 42A and surface 44A. Layer 40B comprises surface 42B and surface 44B. During assembly, layer 40A is positioned above layer 40B such that surface 44A faces surface 44B.

In some embodiments, layer 40A is formed of a plurality of materials with surface 42A comprising an absorbent poly spun bond material (e.g., spunbond polypropylene) and surface 44A comprising a polyethylene film material. In such embodiments, the absorbent poly spun bond material of surface 42A and the polyethylene film material of surface 44A are co-extruded to create layer 40A. It should be appreciated that layer 40A is suitable for use in medical and surgical applications under an AAMI level 4 rating because the porous poly spun bond material of surface 42A is co-extruded with the polyethylene film of surface 44A, which is a hydrophilic compound; thereby forming a sheet of non-linting water-absorbent spun bond material suitable for use in medical and surgical applications and the like.

Layer 40B is substantially the same as layer 40A with surface 42B comprising an absorbent poly spun bond material (e.g., spunbond polypropylene) and surface 44B comprising a polyethylene film material. In such embodiments, the absorbent poly spun bond material of surface 42B and the polyethylene film material of surface 44B are co-extruded to create layer 40B. It should be appreciated that layer 40B is suitable for use in medical and surgical applications under an AAMI level 4 rating because the porous poly spun bond material of surface 42B is co-extruded with the polyethylene film of surface 44B, which is a hydrophilic compound; thereby forming a sheet of non-linting water-absorbent spun bond material suitable for use in medical and surgical applications and the like.

Once properly aligned, layer 40A is placed atop of layer 40B such that surface 44A abuts against or is arranged substantially proximate to surface 44B. Material assembly 40 is arranged on base 20 with surface 42B abutting against or arranged in close proximity with platen 22.

FIG. 3B is a perspective view of material assembly 40. As shown, layer 40A is arranged atop of layer 40B. Sealing bar 50 is displaced in axial direction AD1 toward material assembly 50 until surface 56 engages surface 42A. Once engaged, a predetermined pressure is applied to sealing bar 50. Additionally, sealing bar 50 is heated to a predetermined temperature.

The pressure and temperature of sealing bar 50 results in seal 46 being formed between layer 40A and layer 40B, as shown in FIGS. 3C-D. Seal 46 comprises a permeability rating of the constituent material. Material assembly 40, as shown in FIGS. 3C-D exhibits uniform impenetrability at all points, include at seal 46. Seal 46 as well as layers 40A-B are intended to be impenetrable to viral vectors, proteins, and blood and thus have a “Level 4” rating by AAMI.

It will be appreciated that various aspects of the disclosure above and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the following claims.

### REFERENCE NUMERALS

10 Press assembly
12 Frame
20 Base
22 Platen
24 Teflon layer
26 Teflon layer
30 Ram
32 Platen
34A Column
34B Column
40 Material assembly
40A Layer
40B Layer
42A Surface
42B Surface
44A Surface
44B Surface
46 Seal
50 Sealing bar
52 Component
54 Component
56 Surface
AD1 Axial direction
AD2 Axial direction
RD1 Radial direction
D Distance
W1 Width
W2 Width
H1 Height
H2 Height
R1 Radius
R2 Radius
R3 Radius
R4 Radius

What is claimed is:

1. A sealing bar for creating an Association for the Advancement of Medical Instrumentation (AAMI) rated seal between a first layer of AAMI rated material and a second layer of AAMI rated material, comprising:
a first component including a first width and a first height;
a second component connected to the first component, the second component including:
a second width;
a second height; and,
an engaging surface;
wherein the engaging surface is operatively arranged to engage the first and second layers of AAMI rated materials and form the seal therebetween.

2. The sealing bar as recited in claim 1, wherein the engaging surface is at least partially planar.

3. The sealing bar as recited in claim 2, wherein the sealing bar further comprises at least one radius arranged adjacent the engaging surface.

4. The sealing bar as recited in claim 2, wherein the sealing bar further comprises:
a first radius arranged adjacent the engaging surface; and,
a second radius arranged adjacent the engaging surface and opposite the second width from the first radius.

5. The sealing bar as recited in claim 4, wherein at least one of the first radius and the second radius is 0.030 inches.

6. The sealing bar as recited in claim 4, wherein the second radius is equal to the first radius.

7. The sealing bar as recited in claim 1, wherein the second component is arranged perpendicular to the first component.

8. The sealing bar as recited in claim 7, wherein the sealing bar further comprises at least one radius arranged between the first component and the second component.

9. The sealing bar as recited in claim 8, wherein the sealing bar further comprises:
a third radius arranged between the first component and the second component; and,
a fourth radius arranged between the first component and the second component and opposite the second width from the third radius.

10. The sealing bar as recited in claim 9, wherein at least one of the third radius and the fourth radius is 0.375 inches.

11. The sealing bar as recited in claim 9, wherein the fourth radius is equal to the third radius.

12. A method of connecting a first layer of Association for the Advancement of Medical Instrumentation (AAMI) rated material and a second layer of AAMI rated material, the method comprising:
arranging the first layer of AAMI rated material on a first platen, the first layer of AAMI rated material including a first surface arranged facing toward the first platen and a second surface arranged facing away from the first platen;
arranging the second layer of AAMI rated material on the first layer of AAMI rated material, the second layer of AAMI rated material including a third surface arranged facing toward the second surface and a fourth surface facing away from the second surface;
engaging a sealing bar with the first and second layers of AAMI rated material, the sealing bar comprising an engaging surface arranged to contact the fourth surface;
applying a preset amount of pressure to the sealing bar;
heating the sealing bar to a predetermined temperature; and,
forming an AAMI rated seal between the first layer of AAMI rated material and the second layer of AAMI rated material.

13. The method as recited in claim 12, wherein the sealing bar further comprises:
a first component including a first width and a first height; and,
a second component connected to the first component, the second component including a second width and a second height;
wherein the engaging surface is arranged on the second component.

14. The method as recited in claim 13, wherein the sealing bar further comprises:
a first radius arranged adjacent the engaging surface; and,
a second radius arranged adjacent the engaging surface and opposite the second width from the first radius.

15. The method as recited in claim 12, wherein the predetermined temperature is in the range of 335 to 445 degrees Fahrenheit.

16. The method as recited in claim 12, wherein:
the first and fourth surfaces comprise a poly spun bond material; and,
the second and third surfaces comprise a polyethylene film.

17. The method as recited in claim 16, wherein:
the first and second surfaces are co-extruded to form the first layer; and,
the third and fourth surfaces are co-extruded to form the second layer.

18. The method as recited in claim 12, further comprising, after the step of forming the AAMI rated seal, disengaging the sealing bar from the first and second layers of AAMI rated material after a predetermined period of time.

19. The method as recited in claim 12, wherein the sealing bar further comprises:
a third radius arranged between the first component and the second component; and,
a fourth radius arranged between the first component and the second component and opposite the second width from the third radius.

20. An Association for the Advancement of Medical Instrumentation (AAMI) rated surgical gown or drape comprising at least a first layer of AAMI rated material and a second layer of AAMI rated material, the AAMI rated surgical gown or drape formed using the steps of:
arranging the first layer of AAMI rated material on a first platen, the first layer of AAMI rated material including a first surface arranged facing toward the first platen and a second surface arranged facing away from the first platen;
arranging the second layer of AAMI rated material on the first layer of AAMI rated material, the second layer of AAMI rated material including a third surface arranged facing toward the second surface and a fourth surface facing away from the second surface;
engaging a sealing bar with the first and second layers of AAMI rated material, the sealing bar comprising an engaging surface arranged to contact the fourth surface;
applying a preset amount of pressure to the sealing bar;
heating the sealing bar to a predetermined temperature; and,
forming an AAMI rated seal between the first layer of AAMI rated material and the second layer of AAMI rated material.
